(19) United States
(12) Patent Application Publication
(10) Pub. No.: US 2008/0051558 A1
(43) Pub. Date: Feb. 28, 2008

(54) Method of preparing bivalirudin

(75) Inventor: Yiming Zhou, Shanghai (CN)

Correspondence Address: Raymond Y. Chan, Suite 128, 108 N. Ynez Avenue, Monterey Park, CA, 91754, US

(73) Assignees: Yiming Zhou; Shanghai Soho-Yiming Pharmaceuticals Co., Ltd.

(21) Appl. No.: 11/716,124

(22) Filed: Mar. 9, 2007

(30) Foreign Application Priority Data
Mar. 10, 2006 (CN) 200610024611.5

(51) Int. Cl. C07K 1/04

(52) U.S. Cl. 530/334

(57) ABSTRACT

The present invention relates to a novel solid phase peptide synthesis method for Bivalirudin. This method contains following steps: serving Trityl Chloride Resin, 4-Methyltrityl Chloride Resin, 4-Methoxytrityl Chloride Resin, or 2-Cl Trityl Chloride Resin, or attaching of Wang Resin as a start raw material); according to general solid phase peptide synthesis rules, coupling protected amino acids after deprotection of Fmoc-protection group and then deprotecting side chain protection group; cleaving peptides from resin; and then obtaining crude Bivalirudin product. C18 high pressure liquid chromatography (HPLC) column is applied to purify the product of Bivalirudin. This method is suitable and effective for mass production, in addition to its features of high quality, low production cost, high synthetic yield, avoidance of usage of fatal toxic chemical such as HF, and less environmental pollution. The high yield rate of 99% is achieved for each synthetic step and total yield rate is 14%.

### BACKGROUND OF THE PRESENT INVENTION

1. Field of Invention

This invention relates to Bivalirudin and more particularly to a solid phase peptide synthesis method of producing Bivalirudin (including Acetate, Trifluoroacetate and free base).

2. Description of Related Arts

Bivalirudin is marketed under the brand name: Angiomax® and has a structural formula: D-Phe-Pro-Arg-Pro-Gly-Gly-Gly-Gly-Asn-Gly-Asp-Phe-Glu-Glu-Ile-Pro-Glu-Glu-Tyr-Leu-OH. Its molecular formula and molecular weight is C₉₈H₁₃₈N₂₄O₃₃ and 2178.99.

Medicines Corporation announced to market its new anticoagulation drug Bivalirudin on 10 Nov. 2004. The brand name in the USA is Angiomax®. Currently, it has been approved to be marketed in Europe, the United States, Canada, Israel, New Zealand, Argentina, and etc. Angiomax is a direct thrombin inhibitor. In clinical trial, Bivalirudin could both reduce risk of bleeding and ischematic complications due to the anticoagulative activities of heparin. This advantage also applies to patient with high risk level.

U.S. Pat. No. 5,196,404 discloses a method to produce Bivalirudin. This method associates to Boc protection group. TFA is required to cleave Boc protection group and highly toxic anhydrous HF is required to cleave the peptide from resin. This highly toxic method restricts the Bivalirudin from mass production and use.

### SUMMARY OF THE PRESENT INVENTION

A main objective of the present invention is to provide a method of preparing Bivalirudin by solid phase synthesis.

In order to accomplish the above objective, the present invention provides a method of producing Bivalirudin which includes the following steps:

(a) providing a starting resin which is selected from the group consisting of Trityl Chloride Resin, 4-Methyltrityl Chloride Resin, 4-Methoxytrityl Chloride Resin, 2-Cl Trityl Chloride Resin, or Wang Resin;

(b) coupling protected amino acids according to their order and rules of solid phase peptide synthesis after deprotecting Fmoc-protecting group so as to obtain the protected 20-amino acid of the resin, wherein a coupling reagent is selected from TBTU/HOBT, HBTU/HOBT, BOP/HOBT, TBTU/HOAT, HBTU/HOAT or BOP/HOAT and is used for condensation reaction for deprotecting Fmoc-protecting group;

(c) cleaving and deprotecting side-chain protecting group after obtaining the protected 20-amino acid of the resin to obtain a crude product of Bivalirudin;

(d) purifying the crude product of Bivalirudin by applying C18 (or C8) high pressure liquid chromatography (HPLC) column to obtain a purified product of Bivalirudin; and

(e) freeze drying the purified product to obtain acetate, TFA, or free base of Bivalirudin.

### DETAILED DESCRIPTION OF THE PREFERRED EMBODIMENT

According to the preferred embodiment of the present invention, a method of preparing Bivalirudin includes the steps of:

(a) providing a starting resin which is selected from the group consisting of Trityl Chloride Resin, 4-Methyltrityl Chloride Resin, 4-Methoxytrityl Chloride Resin, 2-Cl Trityl Chloride Resin, or Wang Resin;

(b) coupling protected amino acids according to their order and rules of solid phase peptide synthesis after deprotecting Fmoc-protecting group so as to obtain the protected 20-amino acid of the resin, wherein a coupling reagent is selected from TBTU/HOBT, HBTU/HOBT, BOP/HOBT, TBTU/HOAT, HBTU/HOAT or BOP/HOAT and is used for condensation reaction for deprotecting Fmoc-protecting group;

(c) cleaving and deprotecting side-chain protecting group after obtaining the protected 20-amino acid of the resin to obtain a crude product of Bivalirudin;

(d) purifying the crude product of Bivalirudin by applying C18 (or C8) high pressure liquid chromatography (HPLC) column to obtain a purified product of Bivalirudin; and

(e) freeze drying the purified product to obtain acetate, TFA, or free base of Bivalirudin.

According to the preferred embodiment of the present invention, the method of coupling protected amino acids according to their order after deprotecting Fmoc-protecting group and obtaining the protected 20-amino acid of the resin is further described below.

Step (1): Preparation of Fmoc-Leu-Resin

The preparation of Fmoc-Leu-Resin comprises the steps consisting of:

(a) obtaining a mixture by soaking one of the starting resin selecting from the group consisting of Trityl Chloride Resin, 4-Methyltrityl Chloride Resin, 4-Methoxytrityl Chloride Resin, or 2-Cl Trityl Chloride Resin or Wang Resin (100˜400) in DMF or DCM for 10 to 60 minutes such that the starting resin is swollen completely, wherein in the mixture the concentration of the resin is 5˜20 ml/g and the substitution value is 0.3˜1.5 mmol/g;

(b) adding DIPEA or DMAP with Fmoc-Leu-OH into the mixture described above and allowing reaction under 10˜50° C. for 0.5˜5 hours;

(c) adding methanol to obtain a solvent, allowing reaction under 10˜50° C. for 0.5˜5 hours and blowing dry by nitrogen gas; and

(d) washing the resin with DMF and blowing dry to obtain a Fmoc-Leu-Resin,

where the Mole number of DIPEA or DMAP is 2˜20 times higher than the Resin, the Mole number of Fmoc-Leu-OH is 2˜5 times higher than the Resin, and the concentration of resin is 0.1˜5 ml/g in the solvent.

Step (2): Preparation of Fmoc-Tyr(tBu)-Leu-Resin

The method of producing Fmoc-Tyr(tBu)-Leu-Resin includes the following steps of:

(a) adding deprotection reagent in the Fmoc-Leu-Resin which is obtained from the step (1) as described above, allowing deprotection reaction under 10˜50° C. for 5˜60 minutes and blowing dry;

(b) washing with DMF and blowing dry;

(c) adding a mixture consisting of Fmoc-Tyr(tBu)-OH and TBTU/HOBT, HBTU/HOBT or BOP/HOBT which is dissolved in peptide coupling reagent, allowing coupling reaction under 20˜30° C. for 0.5˜5 hours and blowing dry; and

(d) washing with DMF and ethanol respectively and blowing dry to obtain a Fmoc-Tyr(tBu)-Leu-Resin,

where the step (2) is under a preferred reaction conditions which is used in the preferred embodiment of the present invention. The preferred reaction conditions are as follows:

the composition to volume ratio of the decoupling reagent: PIP:DMF=1: 2˜5; (same for the steps described below)

the peptide coupling reagent composition to volume ratio is: NMM: DMF=1: 5˜15.

the mole number of Fmoc-Tyr(tBu)-OH is 2˜5 times greater than Resin,

the weight ratio of Fmoc-Leu-Resin and deprotection reagent is 5˜20 ml/g,

the mole number of TBTU/HBTU/BOP is 2˜5 times greater than Resin, and

the mole number of HOBT/HOAT is 2˜5 times greater than Resin.

Step (3): Preparation of Fmoc-Glu(OtBu)-Tyr(tBu)-Leu-Resin

The method of producing Fmoc-Glu(OtBu)-Tyr(tBu)-Leu-Resin includes the following steps of:

(a) adding deprotection reagent in the Fmoc-Tyr(tBu)-Leu-Resin which is obtained from step (2) as described above, allowing deprotection reaction under 10˜50° C. for 5-60 minutes and blowing dry;

(b) washing with DMF and blowing dry;

(c) adding a mixture consisting of Fmoc-Thr(tBu)-OH and TBTU/HOBT, HBTU/HOBT or BOP/HOBT which is dissolved in peptide coupling reagent, allowing coupling reaction under 20˜30° C. for 0.5˜5 hours and blowing dry; and

(d) washing with DMF and ethanol respectively and blowing dry to obtain a Fmoc-Glu(OtBu)-Tyr(tBu)-Leu-Resin,

where the mole number of Fmoc-Thr(tBu)-OH is 2˜5 times greater than the Resin and the above preferred reaction conditions as described in step (2) are applied.

Step (4): Preparation of Fmoc-Glu(OtBu)-Glu(OtBu)-Tyr(tBu)-Leu-Resin

The method of producing Fmoc-Glu(OtBu)-Glu(OtBu)-Tyr(tBu)-Leu-Resin includes the following steps of:

(a) adding deprotection reagent in the Fmoc-Glu(OtBu)-Tyr(tBu)-Leu-Resin which is obtained from step (3) as described above, allowing deprotection reaction under 10˜50° C. for 5˜60 minutes and blowing dry;

(b) washing with DMF and blowing dry;

(c) adding a mixture consisting of Fmoc-Phe-OH and TBTU/HOBt, HBTU/HOBT or BOP/HOBT which is dissolved in peptide coupling reagent, allowing coupling reaction under 20˜30° C. for 0.5˜5 hours, and then blowing dry; and

(d) washing with DMF and ethanol respectively and blowing dry accordingly to obtain Fmoc-Glu(OtBu)-Glu(OtBu)-Tyr(tBu)-Leu-Resin,

where the mole number of Fmoc-Glu(OtBu)-OH is 2˜5 times greater than the Resin, and the above preferred reaction conditions as described in step (2) are applied.

Step (5): Preparation of Fmoc-Pro-Glu(OtBu)-Glu(OtBu)-Tyr(tBu)-Leu-Resin

The method of producing Fmoc-Pro-Glu(OtBu)-Glu(OtBu)-Tyr(tBu)-Leu-Resin includes the following steps of:

(a) adding deprotection reagent in Fmoc-Glu(OtBu)-Glu(OtBu)-Tyr(tBu)-Leu-Resin which is obtained from step (4) as described above, allowing deprotection reaction under 10˜50° C. for 5˜60 minutes and blowing dry;

(b) washing with DMF and blowing dry;

(c) adding a mixture consisting of Fmoc-Pro-OH and TBTU/HOBT, HBTU/HOBT or BOP/HOBT which is dissolved in peptide coupling reagent, allowing reaction under 20˜30° C. for 0.5˜5 hours and blowing dry; and

(d) washing with DMF and ethanol respectively and blowing dry accordingly to obtain Fmoc-Pro-Glu(OtBu)-Glu(OtBu)-Tyr(tBu)-Leu-Resin,

where the mole number of Fmoc-Pro-OH is 2˜5 times greater than the Resin and the above preferred reaction conditions as described in step (2) are applied.

Step (6): Preparation of Fmoc-Ile-Pro-Glu(OtBu)-Glu(OtBu)-Tyr(tBu)-Leu-Resin

The method of producing Fmoc-Ile-Pro-Glu(OtBu)-Glu(OtBu)-Tyr(tBu)-Leu-Resin includes the following steps of:

(a) adding deprotection reagent in the Fmoc-Pro-Glu(OtBu)-Glu(OtBu)-Tyr(tBu)-Leu-Resin which is obtained from step (5) as described above, allowing deprotection reaction under 10˜50° C. for 5˜60 minutes and blowing dry;

(b) washing with DMF and blowing dry;

(c) adding a mixture consisting of Fmoc-Lys(Boc)-OH and TBTU/HOBT, HBTU/HOBT or BOP/HOBT which is dissolved in peptide coupling reagent, allowing coupling reaction under 20˜30° C. for 0.5˜5 hours and blowing dry;

(d) washing with DMF and ethanol respectively and blowing dry accordingly to obtain Fmoc-Ile-Pro-Glu(OtBu)-Glu(OtBu)-Tyr(tBu)-Leu-Resin,

where the mole number of Fmoc-Lys(Boc)-OH is 2˜5 times greater than the Resin and the above preferred reaction conditions as described in step (2) are applied.

Step (7): Preparation of Fmoc-Glu(OtBu)-Ile-Pro-Glu(OtBu)-Glu(OtBu)-Tyr(tBu)-Leu-Resin

The method of producing Fmoc-Glu(OtBu)-Ile-Pro-Glu(OtBu)-Glu(OtBu)-Tyr(tBu)-Leu-Resin includes the following steps of:

(a) adding deprotection reagent in Fmoc-Ile-Pro-Glu(OtBu)-Glu(OtBu)-Tyr(tBu)-Leu-Resin as obtained by method (6) and allowing reaction under 10˜50° C. for 5˜60 minutes;

(b) washing with DMF and blowing dry;

(c) adding a mixture consisting of Fmoc-Glu(OtBu)-OH; TBTU/HOBT or HBTU/HOBT or BOP/HOBT which is dissolved in peptide coupling reagent, allowing reaction under 20˜30° C. for 0.5˜5 hours and blowing dry; and

(d) washing with DMF and ethanol respectively and blowing dry accordingly to obtain Fmoc-Glu(OtBu)-Ile-Pro-Glu(OtBu)-Glu(OtBu)-Tyr(tBu)-Leu-Resin,

where the mole number of Fmoc-Glu(OtBu)-OH is 2˜5 times greater than the Resin and the above preferred reaction conditions as described in step (2) are applied.

Step (8): Preparation of Fmoc-Glu(OtBu)-Glu(OtBu)-Ile-Pro-Glu(OtBu)-Glu(OtBu)-Tyr(tBu)-Leu-Resin

The method of producing Fmoc-Glu(OtBu)-Glu(OtBu)-Ile-Pro-Glu(OtBu)-Glu(OtBu)-Tyr(tBu)-Leu-Resin includes the steps of:

(a) adding deprotection reagent in Fmoc-Glu(OtBu)-Ile-Pro-Glu(OtBu)-Glu(OtBu)-Tyr(tBu)-Leu-Resin which is obtained from step (7) as described above, allowing deprotection reaction under 10˜50° C. for 5˜60 minutes and blowing dry;

(b) washing with DMF and blowing dry;

(c) adding a mixture consisting of Fmoc-Glu(OtBu)-OH; TBTU/HOBT or HBTU/HOBT or BOP/HOBT which is dissolved in peptide coupling reagent, allowing coupling reaction under 20˜30° C. for 0.5˜5 hours and blowing dry;

(d) washing with DMF and ethanol respectively and blowing dry accordingly to obtain Fmoc-Glu(OtBu)-Glu(OtBu)-Ile-Pro-Glu(OtBu)-Glu(OtBu)-Tyr(tBu)-Leu-Resin,

where the mole number of Fmoc-Glu(OtBu)-OH is 2˜5 times greater than the Resin and the above preferred reaction conditions as described in step (2) are applied.

Step (9): Preparation of Fmoc-Phe-Glu(OtBu)-Glu(OtBu)-Ile-Pro-Glu(OtBu)-Glu(OtBu)-Tyr(tBu)-Leu-Resin

The method of preparing Fmoc-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in Fmoc-Glu(OtBu)-Glu(OtBu)-Ile-Pro-Glu(OtBu)-Glu(OtBu)-Tyr(tBu)-Leu-Resin which is obtained from step (8) as described above, allowing deprotection reaction under 10˜50° C. for 5˜60 minutes and blowing dry;

(b) washing with DMF and blowing dry;

(c) adding a mixture consisting of Fmoc-Phe-OH and TBTU/HOBT, HBTU/HOBT or BOP/HOBT which is dissolved in peptide coupling reagent, allowing coupling reacting under 20˜30° C. for 0.5˜5 hours and blowing dry;

(d) washing with DMF and ethanol respectively and blowing dry accordingly to obtain Fmoc-Phe-Glu(OtBu)-Glu(OtBu)-Ile-Pro-Glu(OtBu)-Glu(OtBu)-Tyr(tBu)-Leu-Resin,

where the mole number of Fmoc-Phe-OH is 2˜5 times greater than the Resin and the above preferred reaction conditions as described in step (2) are applied.

Step (10): Preparation of Fmoc-Asp(OtBu)-Phe-Glu(OtBu)-Glu(OtBu)-Ile-Pro-Glu(OtBu)-Glu(OtBu)-Tyr(tBu)-Leu-Resin

The method of preparing Fmoc-Asp(OtBu)-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in the Fmoc-Phe-Glu(OtBu)-Glu(OtBu)-Ile-Pro-Glu(OtBu)-Glu(OtBu)-Tyr(tBu)-Leu-Resin which is prepared by the step (9) as described above, allowing deprotection reaction under 10˜50° C. for 5˜60 minutes and blowing dry;

(b) washing with DMF and blowing dry;

(c) adding a mixture consisting of Fmoc-Asp(OtBu)-OH and TBTU/HOBT or HBTU/HOBT or BOP/HOBT which is dissolved in peptide coupling reagent, allowing coupling reaction under 20˜30° C. for 0.5˜5 hours and blowing dry;

(d) washing with DMF and ethanol respectively and blowing dry accordingly to obtain Fmoc-Asp(OtBu)-Phe-Glu(OtBu)-Glu(OtBu)-Ile-Pro-Glu(OtBu)-Glu(OtBu)-Tyr(tBu)-Leu-Resin,

where the mole number of Fmoc-Asp(OtBu)-OH is 2˜5 times greater than the Resin and the above preferred reaction conditions as described in step (2) are applied.

Step (11): Preparation of Fmoc-Gly-Asp(OtBu)-Phe-Glu(OtBu)-Glu(OtBu)-Ile-Pro-Glu(OtBu)-Glu(OtBu)-Tyr(tBu)-Leu-Resin

The method of producing Fmoc-Gly-Asp(OtBu)-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in the Fmoc-Asp(OtBu)-Phe-Glu(OtBu)-Glu(OtBu)-Ile-Pro-Glu(OtBu)-Glu(OtBu)-Tyr(tBu)-Leu-Resin which is prepared from step (10) as described above, allowing deprotection reaction under 10˜50° C. for 5-60 minutes and blowing dry;

(b) washing with DMF and blowing dry;

(c) adding a mixture consisting of Fmoc-Gly-OH and TBTU/HOBT, HBTU/HOBT or BOP/HOBT which is dissolved in peptide coupling reagent, allowing coupling reaction under 20˜30° C. for 0.5˜5 hours and blowing dry; and

(d) washing with DMF and ethanol respectively and blowing dry to obtain a Fmoc-Gly-Asp(OtBu)-Phe-Glu(OtBu)-Glu(OtBu)-Ile-Pro-Glu(OtBu)-Glu(OtBu)-Tyr(tBu)-Leu-Resin,

where the mole number of Fmoc-Gly-OH is 2˜5 times greater than the Resin and the above preferred reaction conditions as described in step (2) are applied.

Step (12): Preparation of Fmoc-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

The method of producing Fmoc-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in the Fmoc-Gly-Asp(OtBu)-Phe-Glu(OtBu)-Glu(OtBu)-Ile-Pro-Glu(OtBu)-Glu(OtBu)-Tyr(tBu)-Leu-Resin which is obtained from step (11) as described above, allowing deprotection reaction under 10˜50° C. for 5-60 minutes and blowing dry;

(b) washing with DMF and blowing dry;

(c) adding a mixture consisting of Fmoc-Asn(Trt)-OH, TBTU/HBTU/BOP and HOBT/HOAT which is dissolved in peptide coupling reagent, allowing coupling reaction under 20˜30° C. for 0.5˜5 hours and blowing dry; and

(d) washing with DMF and ethanol respectively and blowing dry to obtain a Fmoc-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,

where the mole number of Fmoc-Asn(Trt)-OH is 2˜5 times greater than the Resin and the above preferred reaction conditions as described in step (2) are applied.

Step (13): Preparation of Fmoc-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

The method of producing Fmoc-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in the Fmoc-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which is prepared from step (12) as described above, allowing deprotection reaction under 10˜50° C. for 5-60 minutes and blowing dry;

(b) washing with DMF and blowing dry;

(c) adding a mixture consisting of Fmoc-Gly-OH and TBTU/HOBT, HBTU/HOBT or BOP/HOBT which is dissolved in peptide coupling reagent, allowing coupling reaction under 20˜30° C. for 0.5˜5 hours and blowing dry; and

(d) washing with DMF and ethanol respectively and blowing dry to obtain a Fmoc-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,

where the mole number of Fmoc-Gly-OH is 2˜5 times greater than the Resin and the above preferred reaction conditions as described in step (2) are applied.

Step (14): Preparation of Fmoc-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

The method of producing Fmoc-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in the Fmoc-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which is prepared from step (13) as described above, allowing deprotection reaction under 10˜50° C. for 5-60 minutes and blowing dry;

(b) washing with DMF and blowing dry;

(c) adding a mixture consisting of Fmoc-Gly-OH and TBTU/HOBT, HBTU/HOBT or BOP/HOBT which is dissolved in peptide coupling reagent, allowing coupling reaction under 20˜30° C. for 0.5˜5 hours and blowing dry; and

(d) washing with DMF and ethanol respectively and blowing dry to obtain a Fmoc-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,

where the mole number of Fmoc-Gly-OH is 2˜5 times greater than the Resin and the above preferred reaction conditions as described in step (2) are applied.

Step (15): Preparation of Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

The method of producing Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in the Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which is prepared from step (14) as described above, allowing deprotection reaction under 10˜50° C. for 5-60 minutes and blowing dry;

(b) washing with DMF and blowing dry;

(c) adding a mixture consisting of Fmoc-Gly-OH and TBTU/HOBT, HBTU/HOBT or BOP/HOBT which is dissolved in peptide coupling reagent, allowing coupling reaction under 20˜30° C. for 0.5˜5 hours and blowing dry; and

(d) washing with DMF and ethanol respectively and blowing dry to obtain a Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,

where the mole number of Fmoc-Gly-OH is 2˜5 times greater than the Resin and the above preferred reaction conditions as described in step (2) are applied.

Step (16): Preparation of Fmoc-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

The method of producing Fmoc-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in the Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which is prepared from step (15) as described above, allowing deprotection reaction under 10˜50° C. for 5-60 minutes and blowing dry;

(b) washing with DMF and blowing dry;

(c) adding a mixture consisting of Fmoc-Gly-OH and TBTU/HOBT, HBTU/HOBT or BOP/HOBT which is dissolved in peptide coupling reagent, allowing coupling reaction under 20˜30° C. for 0.5˜5 hours and blowing dry; and

(d) washing with DMF and ethanol respectively and blowing dry to obtain a Fmoc-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin.

where the mole number of Fmoc-Gly-OH is 2˜5 times greater than the Resin, and the above preferred reaction conditions as described in step (2) are applied.

Step (17): Preparation of Fmoc-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

The method of preparing Fmoc-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in Fmoc-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which is obtained from step (16) as described above, allowing deprotection reaction under 10˜50° C. for 5˜60 minutes and blowing dry;

(b) washing with DMF and blowing dry;

(c) adding a mixture consisting of Fmoc-Pro-OH and TBTU/HOBT, HBTU/HOBT or BOP/HOBT which is dissolved in peptide coupling reagent, allowing reaction under 20˜30° C. for 0.5˜5 hours and blowing dry; and

(d) washing with DMF and ethanol respectively and blowing dry accordingly to obtain a Fmoc-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,

where the mole number of Fmoc-Pro-OH is 2˜5 times greater than the Resin, and the above preferred reaction conditions as described in step (2) are applied.

Step (18): Preparation of Fmoc-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

The method of producing Fmoc-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in Fmoc-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which is obtained from step (17) as described above, allowing deprotection reaction under 10˜50° C. for 5˜60 minutes and blowing dry;

(b) washing with DMF and blowing dry;

(c) adding a mixture consisting of Fmoc-Arg(Pbf)-OH and TBTU/HOBT, HBTU/HOBT or BOP/HOBT which is dissolved in peptide coupling reagent, allowing reaction under 20˜30° C. for 0.5˜5 hours and blowing dry; and

(d) washing with DMF and ethanol respectively and blowing dry accordingly to obtain a Fmoc-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,

where the mole number of Fmoc-Arg(Pbf)-OH is 2˜5 times greater than the Resin and the above preferred reaction conditions as described in step (2) are applied.

Step (19): Preparation of Fmoc-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

The method of producing Fmoc-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in the Fmoc-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which is prepared by step (18) as described above, allowing deprotection reaction under 10˜50° C. for 5˜60 minutes and blowing dry;

(b) washing with DMF and blowing dry;

(c) adding a mixture consisting of Fmoc-Pro-OH and TBTU/HOBT, HBTU/HOBT or BOP/HOBT which is dissolved in peptide coupling reagent, allowing reaction under 20˜30° C. for 0.5˜5 hours and blowing dry; and

(d) washing with DMF and ethanol respectively and blowing dry accordingly to obtain Fmoc-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,

where the mole number of Fmoc-Pro-OH is 2˜5 times greater than the Resin and the above preferred reaction conditions as described in step (2) are applied.

Step (20): Preparation of Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

The method of producing Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin includes the following steps:

(a) adding deprotection reagent in the Fmoc-Pro-Arg(Pbf)-Pro-Gly-Gly-Gly-Asn(Trt)-Gly-Asp(OtBu)-Phe-Glu(OtBu)-Glu(OtBu)-IlePro-Glu(OtBu)-Glu(OtBu)-Tyr(tBu)-Leu-Resin which is obtained from step (19) as described above, allowing deprotection reaction under 10˜50° C. for 5˜60 minutes and blowing dry;

(b) washing with DMF and blowing dry;

(c) adding a mixture consisting of Boc-D-Phe-OH and TBTU/HOBT, HBTU/HOBT or BOP/HOBT which is dissolved in peptide coupling reagent, allowing coupling reaction under 20˜30° C. for 0.5˜5 hours and blowing dry;

(d) washing with DMF and ethanol respectively and blowing dry accordingly to obtain a Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,

where the mole number of Boc-D-Phe-OH is 2˜5 times greater than the Resin and the above preferred reaction conditions as described in step (2) are applied.

Step (21): Preparation of Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-OH

A method of preparing Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-OH includes the steps of:

(a) adding peptide cleavage reagent at −10° C.˜30° C. (TFA/EDT/H2O/TIS=90-95/2-5/2-5/1-5, volume ratio) into the Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which is prepared from step (20), allowing cleavage reaction under 10° C.˜50° C. for 1˜5 hours;

(b) evaporating solvent and conducting filtration under reduced pressure;

(c) adding cold ether slowly for precipitation, collecting the precipitate formed and washing with ether; and

(d) placing the precipitate in a drying container under room temperature, drying under reduced pressure with P₂O₅ for 10˜14 hours to obtain a crude Bivalirudin product,

where the concentration of Resin in peptide cleavage reagent is 5˜50 ml/g.

According to the present invention, the method of purifying the crude Bivalirudin product includes the following steps:

(i) putting the crude Bivalirudin product in acetic acid solution;

(ii) filtering the solution to form a filtered solution;

(iii) letting the filtered solution flowing through C18 or C8 column, where the liquid phase is: 0.01˜0.5M acetic acid, phosphoric acid or Trifluoroacetic acid (TFA): acetonitrile (10-90: 90-10, v/v); the flow rate is 50˜1,500 ml/min; the detection wavelength is 250˜285 nm;

(iv) using liquid chromatography to track and collect the eluent, combining the peak and removing the salt;

(v) lyophlizing the solution to obtain the purified product of Bivalirudin (MW:2179), where the total yield rate is 14% (in mmole of Resin).

The concentration of acetic acid solution is 0.5˜10% by weight and the concentration of crude product of Bivalirudin in acetic acid is 1˜50% by weight.

The present invention has the followings characteristics. This novel method is capable for mass production with high quality and stability. The stable availability of supply of raw material, the fast production rate, the low cost, the less waste, the high synthetic yield, the capability of avoiding using fatal toxic chemicals such as anhydrous hydrogen fluoride (HF), and the low level of environmental pollution are all advantageous.

According to the preferred embodiment of the present invention, C18 (C8) column applied to purify the product and total yield rate is 14%. This Method apply gentle method Fmoc protection group where Fmoc deprotection requires 25% PIP; peptide cleavage requires TFA which avoid using HF. It greatly reduced the waste, and is greatly beneficial to mass chemical production. It is also compatible to the environmental friendly principle.

### Illustration of the Preferred Embodiments

### EXAMPLE 1

Table of the raw materials of the chemicals
used in the present invention:

| No | Material and Reagent Name | Producer |
|---|---|---|
| 1 | Trityl Chloride Resin | Tianjin Nankai Hechecg S&T |
| | | Co., LTD. |
| 2 | 4-Methyltrityl Chloride Resin | Tianjin Nankai Hechecg S&T |
| | | Co., LTD. |
| 3 | 4-Methoxytrityl Chloride Resin | Tianjin Nankai Hechecg S&T |
| | | Co., LTD. |
| 4 | 2-Cl Trityl Chloride Resin | Tianjin Nankai Hechecg S&T |
| | | Co., LTD. |
| 5 | Wang Resin | Tianjin Nankai Hechecg S&T |
| | | Co., LTD. |
| 6 | Fmoc-Asp(OtBu)-OH | Sichuan Sangao Biochemical |
| | | Corporation, LTD. |
| 7 | Fmoc-Glu(OtBu)-OH | Sichuan Sangao Biochemical |
| | | Corporation, LTD. |
| 8 | Fmoc-Pro-OH | Sichuan Sangao Biochemical |
| | | Corporation, LTD. |
| 9 | Fmoc-Gly-OH | Sichuan Sangao Biochemical |
| | | Corporation, LTD. |
| 10 | Fmoc-Ile-OH | Sichuan Sangao Biochemical |
| | | Corporation, LTD. |
| 11 | Fmoc-Leu-OH | Sichuan Sangao Biochemical |
| | | Corporation, LTD. |
| 12 | Fmoc-Tyr(tBu)-OH | Sichuan Sangao Biochemical |
| | | Corporation, LTD. |
| 13 | Fmoc-Phe-OH | Sichuan Sangao Biochemical |
| | | Corporation, LTD. |
| 14 | Fmoc-Arg(Pbf)-OH | Sichuan Sangao Biochemical |
| | | Corporation, LTD. |
| 15 | Boc-D-Phe-OH | Sichuan Sangao Biochemical |
| | | Corporation, LTD. |
| 16 | HOBt (1-Hydroxybenzotriazole) | Sichuan Sangao Biochemical |
| | | Corporation, LTD. |
| 17 | HOAt(1-Hydroxy-7- | Sichuan Sangao Biochemical |
| | azabenzotriazole) | Corporation, LTD. |
| 18 | HBTU(2-(1H-Benzotriazole-1-yl)- | Sichuan Sangao Biochemical |
| | 1,1,3,3-tetramethyluronium | Corporation, LTD. |
| | hexafluorophosphate) | |
| 19 | TBTU(2-(1H-Benzotriazole-1-yl)- | Sichuan Sangao Biochemical |
| | 1,1,3,3-tetramethyluronium | Corporation, LTD. |
| | tetrafluoroborate) | |
| 20 | BOP(Benzotriazole-1-yl)- | Sichuan Sangao Biochemical |
| | 1,1,3,3-tetramethyluronium | Corporation, LTD. |
| | tetrafluoroborate) | |
| 21 | TFA (Trifluoroacetic acid) | Fluka |
| 22 | NMM(N-Methylmorpholone) | Fluka |
| 23 | EDT(1,2-Ethanedithiol) | Fluka |
| 24 | TIS(Triisopropylsilane) | Fluka |
| 25 | AcN(Acetonitri)l | Merck |
| 26 | DMF(Dimethylformamide) | Merck |
| 27 | MeOH(Methanol) | Shanghai Zhenxin Huagong |
| | | Yichan |
| 28 | PIP(Piperdine) | Shanghai Chemical Reagent Co. |
| 29 | DIPEA(N,N′Diisopropyl | Fluka |
| | ethylamine) | |
| 30 | DMAP(4-Dimethylaminopyridine) | Fluka |

In this example 1:

The peptide coupling reagent is NMM: DMF=1:10 by volume.

The deprotection reagent is PIP: DMF=1:3.5 by volume.

Step (1) Preparation of Fmoc-Leu-Resin

In this step 1, weigh 50 g of 2-Cl Trityl Chloride Resin (100-400, 1.0 mmol/g), soak with 500 ml DMF for 30 minutes, add 44 ml of DIPEA, 17.7 g of Fmoc-Leu-OH (FW: 353.4) and allow reaction for 1 hour. Then, add 20 ml of Methanol and allow reaction at 25° C. for 1 hour, suck out the methanol. Lastly, wash the Resin with DMF for six times and blow dry.

Step (2) Preparation of Fmoc-Tyr(tBu)-Leu-Resin

In this step 2, add 500 ml deprotection reagent into the Fmoc-Leu-Resin from step (1), allow reaction under 25° C. for 30 mins and blow dry. Wash with DMF and blow dry. Add a mixture consisting of 68.9 g of Fmoc-Tyr(tBu)-OH (459.6), 48.1 g of TBTU (FW:321.1) and 20.2 g of HOBT (FW:135.1) which is dissolved by using 400 ml of peptide coupling reagent, allow reaction under 25° C. for 2 hours and blow dry. Wash with DMF and ethanol respectively and blow dry to obtain a Fmoc-Tyr(tBu)-Leu-Resin.

Step (3) Preparation of Fmoc-Glu(OtBu)-Tyr(tBu)-Leu-Resin

In this step 3, add 500 ml deprotection reagent into the Fmoc-Tyr(tBu)-Leu-Resin from step (2), allow reaction under 25° C. for 30 mins and blow dry. Wash with DMF and blow dry. Add a mixture consisting of 63.8 g of Fmoc-Glu(OtBu)-OH (FW:425.5), 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Glu(OtBu)-Tyr(tBu)-Leu-Resin, where the mole number of Fmoc-Glu(OtBu)-OH is 2˜5 times greater than the Resin and other conditions are the same as those of the previous step (step (2)).

Step (4): Preparation of Fmoc-Glu(OtBu)-Glu(OtBu)-Tyr(tBu)-Leu-Resin

In this step (4), add 500 ml deprotection reagent into the Fmoc-Glu(OtBu)-Tyr(tBu)-Leu-Resin from step (3), allow reaction under 25° C. for 30 mins and blow dry. Wash with DMF and blow dry. Add a mixture consisting of 63.8 g of Fmoc-Glu (OtBu)-OH(425.5), 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Glu(OtBu)-Glu(OtBu)-Tyr(tBu)-Leu-Resin, where other conditions are the same as those of the previous step (step (2)).

Step (5): Preparation of Fmoc-Pro-Glu(OtBu)-Glu(OtBu)-Tyr(tBu)-Leu-Resin

In this step (5), add 500 ml deprotection reagent into the Fmoc-Glu(OtBu)-Glu(OtBu)-Tyr(tBu)-Leu-Resin from step (4), allow reaction under 25° C. for 30 mins and blow dry. Wash with DMF and blow dry. Add a mixture consisting of 50.6 g of Fmoc-Pro-OH (FW:337.4), 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Pro-Glu(OtBu)-Glu(OtBu)-Tyr(tBu)-Leu-Resin, where other conditions are the same as those of the previous step (step (2)).

Step (6): Preparation of Fmoc-Ile-Pro-Glu(OtBu)-Glu(OtBu)-Tyr(tBu)-Leu-Resin

In this step (6), add 500 ml deprotection reagent into the Fmoc-Pro-Glu(OtBu)-Glu(OtBu)-Tyr(tBu)-Leu-Resin from step (5), allow reaction under 25° C. for 30 mins and blow dry. Wash with DMF and blow dry. Add a mixture consisting of 53.1 g of Fmoc-Ile-OH (FW:353.4), 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Ile-Pro-Glu(OtBu)-Glu(OtBu)-Tyr(tBu)-Leu-Resin, where other conditions are the same as those of the previous step (step (2)).

Step (7): Preparation of Fmoc-Glu(OtBu)-Ile-Pro-Glu(OtBu)-Glu(OtBu)-Tyr(tBu)-Leu-Resin

In this step (7), add 500 ml deprotection reagent into the Fmoc-Ile-Pro-Glu(OtBu)-Glu(OtBu)-Tyr(tBu)-Leu-Resin from step (6), allow reaction under 25° C. for 30 mins and blow dry. Wash with DMF and blow dry. Add a mixture consisting of 63.8 g of Fmoc-Glu(OtBu)-OH, 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Glu(OtBu)-Ile-Pro-Glu(OtBu)-Glu(OtBu)-Tyr(tBu)-Leu-Resin, where other conditions are the same as those of the previous step (step (2)).

Step (8): Preparation of Fmoc-Glu(OtBu)-Glu(OtBu)-Ile-Pro-Glu(OtBu)-Glu(OtBu)-Tyr(tBu)-Leu-Resin

In this step (8), add 500 ml deprotection reagent into the Fmoc-Glu(OtBu)-Ile-Pro-Glu(OtBu)-Glu(OtBu)-Tyr(tBu)-Leu-Resin from step (7), allow reaction under 25° C. for 30 mins and blow dry. Wash with DMF and blow dry. Add a mixture consisting of 63.4 g of Fmoc-Glu(OtBu)-OH, 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (9): Preparation of Fmoc-Phe-Glu(OtBu)-Glu(OtBu)-Ile-Pro-Glu(OtBu)-Glu(OtBu)-Tyr(tBu)-Leu-Resin

In this step (9), add 500 ml deprotection reagent into the Fmoc-Glu(OtBu)-Glu(OtBu)-Ile-Pro-Glu(OtBu)-Glu(OtBu)-Tyr(tBu)-Leu-Resin from step (8), allow reaction under 25° C. for 30 mins and blow dry. Wash with DMF and blow dry. Add a mixture consisting of 58.1 g of Fmoc-Phe-OH (FW:387.4), 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Phe-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (10): Preparation of Fmoc-Asp(OtBu)-Phe-Glu(OtBu)-Glu(OtBu)-Ile-Pro-Glu(OtBu)-Glu(OtBu)-Tyr(tBu)-Leu-Resin

In this step (10), add 500 ml deprotection reagent into the Fmoc-Phe-Glu(OtBu)-Glu(OtBu)-Ile-Pro-Glu(OtBu)-Glu(OtBu)-Tyr(tBu)-Leu-Resin from step (9), allow reaction under 25° C. for 30 mins and blow dry. Wash with DMF and blow dry. Add a mixture consisting of 61.7 g of Fmoc-Asp(OtBu)-OH(FW:411.5), 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Asp(OtBu)-Phe-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (11): Preparation of Fmoc-Gly-Asp(OtBu)-Phe-Glu(OtBu)-Glu(OtBu)-Ile-Pro-Glu(OtBu)-Glu(OtBu)-Tyr(tBu)-Leu-Resin

In this step (11), add 500 ml deprotection reagent into the Fmoc-Asp(OtBu)-Phe-Glu(OtBu)-Glu(OtBu)-Ile-Pro-Glu(OtBu)-Glu(OtBu)-Tyr(tBu)-Leu-Resin from step (10), allow reaction under 25° C. for 30 mins and blow dry. Wash with DMF and blow dry. Add a mixture consisting of 44.6 g of Fmoc-Gly-OH (FW:297.3), 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Gly-Asp(OtBu)-Phe-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (12): Preparation of Fmoc-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

In this step (12), add 500 ml deprotection reagent into the Fmoc-Gly-Asp(OtBu)-Phe-Glu(OtBu)-Glu(OtBu)-Ile-Pro-Glu(OtBu)-Glu(OtBu)-Tyr(tBu)-Leu-Resin from step (11), allow reaction under 25° C. for 30 mins and blow dry. Wash with DMF and blow dry. Add a mixture consisting of 89.5 g of Fmoc-Asn(Trt)-OH(FW:596.7), 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (13): Preparation of Fmoc-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

In this step (13), add 500 ml deprotection reagent into the Fmoc-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (12), allow reaction under 25° C. for 30 mins and blow dry. Wash with DMF and blow dry. Add a mixture consisting of 44.6 g of Fmoc-Gly-OH, 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (14): Preparation of Fmoc-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin In this step (14), add 500 ml deprotection reagent into the Fmoc-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (13), allow reaction under 25° C. for 30 mins and blow dry. Wash with DMF and blow dry. Add a mixture consisting of 44.6 g of Fmoc-Gly-OH, 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (15): Preparation of Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

In this step (15), add 500 ml deprotection reagent into the Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (14), allow reaction under 25° C. for 30 mins and blow dry. Wash with DMF and blow dry. Add a mixture consisting of 44.6 g of Fmoc-Gly-OH, 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (16): Preparation of Fmoc-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

In this step (16), add 500 ml deprotection reagent into the Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (15), allow reaction under 25° C. for 30 mins and blow dry. Wash with DMF and blow dry. Add a mixture consisting of 44.6 g of Fmoc-Gly-OH, 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (17): Preparation of Fmoc-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

In this step (17), add 500 ml deprotection reagent into the Fmoc-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (16), allow reaction under 25° C. for 30 mins and blow dry. Wash with DMF and blow dry. Add a mixture consisting of 50.6 g of Fmoc-Pro-OH (FW:337.4), 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (18): Preparation of Fmoc-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

In this step (18), add 500 ml deprotection reagent into the Fmoc-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (17), allow reaction under 25° C. for 30 mins and blow dry. Wash with DMF and blow dry. Add a mixture consisting of 97.3 g of Fmoc-Arg(Pbf)-OH, 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (19): Preparation of Fmoc-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

In this step (19), add 500 ml deprotection reagent into the Fmoc-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (18), allow reaction under 25° C. for 30 mins and blow dry. Wash with DMF and blow dry. Add a mixture consisting of 50.6 g of Fmoc-Pro-OH, 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Fmoc-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin, where other conditions are the same as those of the previous step (step (2)).

Step (20): Preparation of Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin

In this step (20), add 500 ml deprotection reagent into the Fmoc-Pro-Arg(Pbf)-Pro-Gly-Gly-Gly-Asn(Trt)-Gly-Asp(OtBu)-Phe-Glu(OtBu)-Glu(OtBu)-IlePro-Glu(OtBu)-Glu(OtBu)-Tyr(tBu)-Leu-Resin from step (19), allow reaction under 25° C. for 30 mins and blow dry. Wash with DMF and blow dry. Add a mixture consisting of 39.8 g of Boc-D-Phe-OH, 48.1 g of TBTU and 20.2 g of HOBT which is dissolved by using 400 ml of peptide coupling reagent, allow reaction under 25° C. for 2 hours and blow dry. Wash with DMF and ethanol respectively and blow dry to obtain a Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin, wherein the protected 20 amino acid resin obtained is about 130 g and where other conditions are the same as those of the previous step (step (2)).

Step (21): Preparation of D-Phe-Pro-Arg-Pro-Gly-Gly-Gly-Gly-Asn-Phe-Glu-Glu-Ile-Pro-Glu-Glu-Tyr-Leu-OH

This step (21) comprises the followings:

(i) adding peptide cleavage reagent at 0° C. (TFA/EDT/H2O/TIS=1260 ml/56 ml/35 ml/14 ml, by volume) into the Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which is prepared from step (20), allowing cleavage reaction under 25° C. for 3 hours;

(ii) evaporating solvent and conducting filtration under reduced pressure;

(iii) adding 1500 ml of cold ether slowly, collecting the precipitate formed which is then washed with ether; and

(iv) placing the precipitate in a drying container under room temperature, drying under reduced pressure with P₂O₅ for 12 hours to obtain a crude Bivalirudin product which is about 60 g;

(v) putting the crude Bivalirudin product in 5% acetic acid solution;

(vi) filtering the solution to form a filtered solution;

(vii) letting the solution flow through C18 column, wherein the liquid phase is: 0.2M acetate: acetonitrile (7.5: 2.5, by volume); the flow rate is 350 ml/min; the detection wavelength is 280 nm;

(viii) analyzing using the method of liquid chromatography, collecting the eluent, combining the peak and removing the salt; and

(ix) freeze drying the solution obtained from step (viii) to obtain a final product of Bivalirudin which is 15.2 g (MW:2179), wherein the total yield is 14%.

### EXAMPLE 2

In this example 2, the methods and conditions used are the same as those of example 1, except that Wang Resin is used as the starting resin. After the Wang Resin is completely swollen in DMF, Fmoc-Leu-OH, TBTU, HOBT, NMM and DMF are added and the reaction is carried out at 25° C. for 2 hours, and that the resin is washed by DMF and anhydrous ethanol respectively for three times.

Deprotection reagent is then added for reaction for 0.5 hours under 25° C. Then, the resin is fully washed and mixed with a mixture consisting of Fmoc-amino acid, TBTU/HOBt, HBTU/HOBt, BOP/HOBt in dissolved peptide coupling reagent for reaction for 2 hours under 25° C. This process is continued until the completion of coupling of the 20 amino acid of the resin. Finally, 14.6 g white powder of the final product of Bivalirudin in white powder is obtained by deprotection and coupling reaction.

1-10. (canceled)

11. A method of preparing Bivalirudin using solid phase peptide synthesis comprising the steps consisting of:
(i) using a starting resin material which is selected from Trityl Chloride Resin, 4-Methyltrityl Chloride Resin, 4-Methoxytrityl Chloride Resin, 2-Cl Trityl Chloride Resin with the C-terminal amino acid, or Wang Resin;
(ii) coupling protected amino acids after deprotection of Fmoc-protection group according to the rules of solid phase peptide synthesis;
(iii) coupling amino acids by using a reagent selected from a group consisting of TBTU/HOBT, HBTU/HOBT, BOP/HOBT, TBTU/HOAT, HBTU/HOAT, and BOP/HOAT for condensation reaction;
(iv) continuing said step (b) with side chain deprotection group to form an intermediate resin material;
(v) cleaving peptides from said intermediate resin material to obtain a crude product of Bivalirudin; and
(vi) purifying said crude product by applying a C18 or C8 column in high pressure liquid chromatography (HPLC) and then freeze drying to obtain a final product of Bivalirudin.

12. The method, as recited in claim 11, wherein in said step (ii) further includes a method of coupling protected amino acids after deprotection of Fmoc-protection group according to the rules of solid phase peptide synthesis consisting the steps of:
(a) preparing Fmoc-Leu-Resin which comprises the steps consisting of:
(a.1) soaking said starting resin in DMF or DCM for 10 to 60 minutes to form a mixture such that said starting resin is fully swollen and the volume to mass ratio of said mixture is 5 to 20 ml/g;
(a.2) adding DIPEA or DMAP in addition to Fmoc-Leu-OH to said mixture and allowing reaction under 10 to 50° C. for 0.5 to 5 hours;
(a.3) adding methanol and obtaining a solvent, then reacting under 10 to 50° C. for 0.5 to 5 hours and blowing dry with nitrogen gas;
(a.4) washing with DMF, blowing dry with nitrogen gas to obtain a first semi product of Fmoc-Leu-Resin,
where the mole number of said DIPEA or DMAP is 2 to 20 times larger than said resin, the mole number of said Fmoc-Leu-OH is 2 to 5 times larger than said resin, and the concentration of said Resin is 0.1 to 5 ml/g in said solvent;
(b) preparing Fmoc-Tyr(tBu)-Leu-Resin which further comprises the steps consisting of:
(b.1) adding a deprotection reagent in said Fmoc-Leu-Resin from the step (a), allowing deprotection reaction and blowing dry;
(b.2) washing with DMF and blowing dry;
(b.3) adding a mixture consisting of (A) TBTU/HOBT, HBTU/HOBT or BOP/HOBT and (B) Fmoc-Tyr(tBu)-OH which are dissolved in peptide coupling reagent, allowing peptide coupling reaction and blowing dry; and
(b.4) washing with DMF and ethanol respectively and blowing dry to obtain a second semi product of Fmoc-Tyr(tBu)-Leu-Resin,
where said step (b) applies a first set of conditions consisting of the component to volume ratio of said deprotection reagent which is: PIP: DMF=1: 2˜5 and the component to volume ratio of said coupling reagent which is: NMM: DMF=1:5˜15, that said first set of conditions are applied for the subsequent steps as described, and
where in said step (b), the mole number of said Fmoc-Tyr(tBu)-OH is 2˜5 times greater than said Resin, the ratio of said Fmoc-Leu-Resin by weight to the quantity of said deprotection reagent is: 5˜20 ml/g, the mole number of said TBTU/HBTU/BOP is 2˜5 times greater than that of said Resin, and the mole number of said HOBT/HOAT is 2˜5 times greater than that of said Resin,
(c) preparing Fmoc-Glu(OtBu)-Tyr(tBu)-Leu-Resin which further comprises the steps consisting of:
(c.1) adding a deprotection reagent in said Fmoc-Tyr(tBu)-Leu-Resin from step (b), allowing deprotection reaction and blowing dry;
(c.2) washing with DMF and blowing dry;
(c.3) adding a mixture consisting of (A) TBTU/HOBT, HBTU/HOBT or BOP/HOBT and (B) Fmoc-Glu(OtBu)-OH which are dissolved in peptide coupling reagent, allowing peptide coupling reaction and blowing dry; and
(c.4) washing with DMF and ethanol respectively and blowing dry to obtain a third semi product of Fmoc-Glu(OtBu)-Tyr(tBu)-Leu-Resin,
where in said step (c), the mole number of said Fmoc-Glu(OtBu)-OH is 2˜5 times greater than that of said Resin and said first set of conditions as described in step (b) applies;
(d) preparing Fmoc-Glu(OtBu)-Glu(OtBu)-Tyr(tBu)-Leu-Resin which further comprises the steps consisting of:
(d.1) adding a deprotection reagent in said Fmoc-Glu(OtBu)-Tyr(tBu)-Leu-Resin from the step (c), allowing deprotection reaction and blowing dry;
(d.2) washing with DMF and blowing dry;
(d.3) adding a mixture consisting of (A) TBTU/HOBT, HBTU/HOBT or BOP/HOBT and (B) Fmoc-Glu(OtBu)-OH which are dissolved in peptide coupling reagent, allowing peptide coupling reaction and blowing dry; and
(d.4) washing with DMF and ethanol respectively and blowing dry to obtain a fourth semi product of Fmoc-Glu(OtBu)-Glu(OtBu)-Tyr(tBu)-Leu-Resin,
where in said step (d), the mole number of said Fmoc-Glu(OtBu)-OH is 2˜5 times greater than that of said Resin and said first set of conditions as described in step (b) applies;
(e) preparing Fmoc-Pro-Glu(OtBu)-Glu(OtBu)-Tyr(tBu)-Leu-Resin which further comprises the steps consisting of:
(e.1) adding a deprotection reagent in said Fmoc-Glu(OtBu)-Glu(OtBu)-Tyr(tBu)-Leu-Resin from step (d), allowing deprotection reaction and blowing dry;
(e.2) washing with DMF and blowing dry;
(e.3) adding a mixture consisting of (A) TBTU/HOBT, HBTU/HOBT or BOP/HOBT and (B) Fmoc-Pro-OH which are dissolved in peptide coupling reagent, allowing peptide coupling reaction and blowing dry; and
(e.4) washing with DMF and ethanol respectively and blowing dry to obtain a fifth semi product of Fmoc-Pro-Glu(OtBu)-Glu(OtBu)-Tyr(tBu)-Leu-Resin
where in said step (e), the mole number of said Fmoc-Pro-OH is 2˜5 times greater than that of said Resin and said first set of conditions as described in step (b) applies;
(f) preparing Fmoc-Ile-Pro-Glu(OtBu)-Glu(OtBu)-Tyr(tBu)-Leu-Resin which further comprises the steps consisting of:
(f.1) adding a deprotection reagent in said Fmoc-Pro-Glu(OtBu)-Glu(OtBu)-Tyr(tBu)-Leu-Resin from step (e), allowing deprotection reaction and blowing dry;
(f.2) washing with DMF and blowing dry;
(f.3) adding a mixture consisting of (A) TBTU/HOBT, HBTU/HOBT or BOP/HOBT and (B) Fmoc-Ile-OH which are dissolved in peptide coupling reagent, allowing peptide coupling reaction and blowing dry; and
(f.4) washing with DMF and ethanol respectively and blowing dry to obtain a sixth semi product of Fmoc-Ile-Pro-Glu(OtBu)-Glu(OtBu)-Tyr(tBu)-Leu-Resin,
where in said step (f), the mole number of said Fmoc-Ile-OH is 2˜5 times greater than that of said Resin and said first set of conditions as described in step (b) applies;
(g) preparing Fmoc-Glu(OtBu)-Ile-Pro-Glu(OtBu)-Glu(OtBu)-Tyr(tBu)-Leu-Resin which further comprises the steps consisting of:
(g.1) adding a deprotection reagent in said Fmoc-Ile-Pro-Glu(OtBu)-Glu(OtBu)-Tyr(tBu)-Leu-Resin from step (f), allowing deprotection reaction and blowing dry;
(g.2) washing with DMF and blowing dry;
(g.3) adding a mixture consisting of (A) TBTU/HOBT, HBTU/HOBT or BOP/HOBT and (B) Fmoc-Glu(OtBu)-OH which are dissolved in peptide coupling reagent, allowing peptide coupling reaction and blowing dry; and
(g.4) washing with DMF and ethanol respectively and blowing dry to obtain a seventh semi product of Fmoc-Glu(OtBu)-Ile-Pro-Glu(OtBu)-Glu(OtBu)-Tyr(tBu)-Leu-Resin,
where in said step (g), the mole number of said Fmoc-Glu(OtBu)-OH is 2˜5 times greater than that of said Resin and said first set of conditions as described in step (b) applies;
(h) preparing Fmoc-Glu(OtBu)-Glu(OtBu)-Ile-Pro-Glu(OtBu)-Glu(OtBu)-Tyr(tBu)-Leu-Resin which further comprises the steps consisting of:
(h.1) adding a deprotection reagent in said Fmoc-Glu(OtBu)-Ile-Pro-Glu(OtBu)-Glu(OtBu)-Tyr(tBu)-Leu-Resin from step (g), allowing deprotection reaction and blowing dry;
(h.2) washing with DMF and blowing dry;
(h.3) adding a mixture consisting of (A) TBTU/HOBT, HBTU/HOBT or BOP/HOBT and (B) Fmoc-Glu(OtBu)-OH which are dissolved in peptide coupling reagent, allowing peptide coupling reaction and blowing dry; and
(h.4) washing with DMF and ethanol respectively and blowing dry to obtain an eighth semi product of Fmoc-Glu(OtBu)-Glu(OtBu)-Ile-Pro-Glu(OtBu)-Glu(OtBu)-Tyr(tBu)-Leu-Resin
where in said step (h), the mole number of said Fmoc-Glu(OtBu)-OH is 2˜5 times greater than that of said Resin and said first set of conditions as described in step (b) applies;
(i) preparing Fmoc-Phe-Glu(OtBu)-Glu(OtBu)-Ile-Pro-Glu(OtBu)-Glu(OtBu)-Tyr(tBu)-Leu-Resin which further comprises the steps consisting of:
(i.1) adding a deprotection reagent in said Fmoc-Glu(OtBu)-Glu(OtBu)-Ile-Pro-Glu(OtBu)-Glu(OtBu)-Tyr(tBu)-Leu-Resin from step (h), allowing deprotection reaction and blowing dry;
(i.2) washing with DMF and blowing dry;
(i.3) adding a mixture consisting of (A) TBTU/HOBT, HBTU/HOBT or BOP/HOBT and (B) Fmoc-Phe-OH which are dissolved in peptide coupling reagent, allowing peptide coupling reaction and blowing dry; and
(i.4) washing with DMF and ethanol respectively and blowing dry to obtain a ninth semi product of Fmoc-Phe-Glu(OtBu)-Glu(OtBu)-Ile-Pro-Glu(OtBu)-Glu(OtBu)-Tyr(tBu)-Leu-Resin,
where in said step (i), the mole number of said Fmoc-Phe-OH is 2˜5 times greater than that of said Resin and said first set of conditions as described in step (b) applies;
(j) preparing Fmoc-Asp(OtBu)-Phe-Glu(OtBu)-Glu(OtBu)-Ile-Pro-Glu(OtBu)-Glu(OtBu)-Tyr(tBu)-Leu-Resin which further comprises the steps consisting of:
(j.1) adding a deprotection reagent in said Fmoc-Phe-Glu(OtBu)-Glu(OtBu)-Ile-Pro-Glu(OtBu)-Glu(OtBu)-Tyr(tBu)-Leu-Resin from step (i), allowing deprotection reaction and blowing dry;
(j.2) washing with DMF and blowing dry;
(j.3) adding a mixture consisting of (A) TBTU/HOBT, HBTU/HOBT or BOP/HOBT and (B) Fmoc-Asp(OtBu)-OH which are dissolved in peptide coupling reagent, allowing peptide coupling reaction and blowing dry; and
(j.4) washing with DMF and ethanol respectively and blowing dry to obtain a tenth semi product of Fmoc-Asp(OtBu)-Phe-Glu(OtBu)-Glu(OtBu)-Ile-Pro-Glu(OtBu)-Glu(OtBu)-Tyr(tBu)-Leu-Resin,
where in said step (j), the mole number of said Fmoc-Asp(OtBu)-OH is 2˜5 times greater than that of said Resin and said first set of conditions as described in step (b) applies;
(k) preparing Fmoc-Gly-Asp(OtBu)-Phe-Glu(OtBu)-Glu(OtBu)-Ile-Pro-Glu(OtBu)-Glu(OtBu)-Tyr(tBu)-Leu-Resin which further comprises the steps consisting of:
(k.1) adding a deprotection reagent in said Fmoc-Asp(OtBu)-Phe-Glu(OtBu)-Glu(OtBu)-Ile-Pro-Glu(OtBu)-Glu(OtBu)-Tyr(tBu)-Leu-Resin from step (j), allowing deprotection reaction and blowing dry;
(k.2) washing with DMF and blowing dry;
(k.3) adding a mixture consisting of (A) TBTU/HOBT, HBTU/HOBT or BOP/HOBT and (B) Fmoc-Gly-OH which are dissolved in peptide coupling reagent, allowing peptide coupling reaction and blowing dry; and
(k.4) washing with DMF and ethanol respectively and blowing dry to obtain an eleventh semi product of Fmoc-Gly-Asp(OtBu)-Phe-Glu(OtBu)-Glu(OtBu)-Ile-Pro-Glu(OtBu)-Glu(OtBu)-Tyr(tBu)-Leu-Resin,
where in said step (k), the mole number of said Fmoc-Gly-OH is 2˜5 times greater than that of said Resin and said first set of conditions as described in step (b) applies;
(l) preparing Fmoc-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which further comprises the steps consisting of:
(l.1) adding a deprotection reagent in said Fmoc-Gly-Asp(OtBu)-Phe-Glu(OtBu)-Glu(OtBu)-Ile-Pro-Glu(OtBu)-Glu(OtBu)-Tyr(tBu)-Leu-Resin from step (k), allowing deprotection reaction and blowing dry;
(l.2) washing with DMF and blowing dry;
(l.3) adding a mixture consisting of (A) TBTU/HOBT, HBTU/HOBT or BOP/HOBT and (B) Fmoc-Asn(Trt)-OH which are dissolved in peptide coupling reagent, allowing peptide coupling reaction and blowing dry; and
(l.4) washing with DMF and ethanol respectively and blowing dry to obtain a twelfth semi product of Fmoc-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,
where in said step (l), the mole number of said Fmoc-Asn(Trt)-OH is 2˜5 times greater than that of said Resin and said first set of conditions as described in step (b) applies;
(m) preparing Fmoc-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which further comprises the steps consisting of:
(m.1) adding a deprotection reagent in said Fmoc-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (l), allowing deprotection reaction and blowing dry;
(m.2) washing with DMF and blowing dry;
(m.3) adding a mixture consisting of (A) TBTU/HOBT, HBTU/HOBT or BOP/HOBT and (B) Fmoc-Gly(Trt)-OH which are dissolved in peptide coupling reagent, allowing peptide coupling reaction and blowing dry; and
(m.4) washing with DMF and ethanol respectively and blowing dry to obtain a thirteenth semi product of Fmoc-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,
where in said step (m), the mole number of said Fmoc-Gly(Trt)-OH is 2˜5 times greater than that of said Resin and said first set of conditions as described in step (b) applies;
(n) preparing Fmoc-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which further comprises the steps consisting of:
(n.1) adding a deprotection reagent in said Fmoc-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (m), allowing deprotection reaction and blowing dry;
(n.2) washing with DMF and blowing dry;
(n.3) adding a mixture consisting of (A) TBTU/HOBT, HBTU/HOBT or BOP/HOBT and (B) Fmoc-Gly-OH which are dissolved in peptide coupling reagent, allowing peptide coupling reaction and blowing dry; and
(n.4) washing with DMF and ethanol respectively and blowing dry to obtain a fourteenth semi product of Fmoc-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,
where in said step (n), the mole number of said Fmoc-Gly-OH is 2˜5 times greater than that of said Resin and said first set of conditions as described in step (b) applies;
(o) preparing Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which further comprises the steps consisting of:
(o.1) adding a deprotection reagent in said Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (n), allowing deprotection reaction and blowing dry;
(o.2) washing with DMF and blowing dry;
(o.3) adding a mixture consisting of (A) TBTU/HOBT, HBTU/HOBT or BOP/HOBT and (B) Fmoc-Gly-OH which are dissolved in peptide coupling reagent, allowing peptide coupling reaction and blowing dry; and
(o.4) washing with DMF and ethanol respectively and blowing dry to obtain a fifteenth semi product of Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,
where in said step (o), the mole number of said Fmoc-Gly-OH is 2˜5 times greater than that of said Resin and said first set of conditions as described in step (b) applies;
(p) preparing Fmoc-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which further comprises the steps consisting of:
(p.1) adding a deprotection reagent in said Fmoc-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (o), allowing deprotection reaction and blowing dry;
(p.2) washing with DMF and blowing dry;
(p.3) adding a mixture consisting of (A) TBTU/HOBT, HBTU/HOBT or BOP/HOBT and (B) Fmoc-Gly-OH which are dissolved in peptide coupling reagent, allowing peptide coupling reaction and blowing dry; and
(p.4) washing with DMF and ethanol respectively and blowing dry to obtain a sixteenth semi product of Fmoc-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,
where in said step (p), the mole number of said Fmoc-Gly-OH is 2˜5 times greater than that of said Resin and said first set of conditions as described in step (b) applies;
(q) preparing Fmoc-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which further comprises the steps consisting of:
(q.1) adding a deprotection reagent in said Fmoc-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (p), allowing deprotection reaction and blowing dry;
(q.2) washing with DMF and blowing dry;
(q.3) adding a mixture consisting of (A) TBTU/HOBT, HBTU/HOBT or BOP/HOBT and (B) Fmoc-Pro-OH which are dissolved in peptide coupling reagent, allowing peptide coupling reaction and blowing dry; and
(q.4) washing with DMF and ethanol respectively and blowing dry to obtain a seventeenth semi product of Fmoc-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,
where in said step (q), the mole number of said Fmoc-Pro-OH is 2˜5 times greater than that of said Resin and said first set of conditions as described in step (b) applies;
(r) preparing Fmoc-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which further comprises the steps consisting of:
(r.1) adding a deprotection reagent in said Fmoc-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (q), allowing deprotection reaction and blowing dry;
(r.2) washing with DMF and blowing dry;
(r.3) adding a mixture consisting of (A) TBTU/HOBT, HBTU/HOBT or BOP/HOBT and (B) Fmoc-Arg(Pbf)-OH which are dissolved in peptide coupling reagent, allowing peptide coupling reaction and blowing dry; and
(r.4) washing with DMF and ethanol respectively and blowing dry to obtain a third semi product of Fmoc-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,
where in said step (r), the mole number of said Fmoc-Arg(Pbf)-OH is 2˜5 times greater than that of said Resin and said first set of conditions as described in step (b) applies;
(s) preparing Fmoc-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which further comprises the steps consisting of:
(s.1) adding a deprotection reagent in said Fmoc-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from step (r), allowing deprotection reaction and blowing dry;
(s.2) washing with DMF and blowing dry;
(s.3) adding a mixture consisting of (A) TBTU/HOBT, HBTU/HOBT or BOP/HOBT and (B) Fmoc-Pro-OH which are dissolved in peptide coupling reagent, allowing peptide coupling reaction and blowing dry; and
(s.4) washing with DMF and ethanol respectively and blowing dry to obtain a nineteenth semi product of Fmoc-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,
where in said step (s), the mole number of said Fmoc-Pro-OH is 2˜5 times greater than that of said Resin and said first set of conditions as described in step (b) applies;
(t) preparing Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin which further comprises the steps consisting of:
(t.1) adding a deprotection reagent in said Fmoc-Pro-Arg(Pbf)-Pro-Gly-Gly-Gly-Asn(Trt)-Gly-Asp(OtBu)-Phe-Glu(OtBu)-Glu(OtBu)-IlePro-Glu(OtBu)-Glu(OtBu)-Tyr(tBu)-Leu-Resin from said step (s), allowing deprotection reaction and blowing dry;
(t.2) washing with DMF and blowing dry;
(t.3) adding a mixture consisting of (A) TBTU/HOBT, HBTU/HOBT or BOP/HOBT and (B) Boc-D-Phe-OH which are dissolved in peptide coupling reagent, allowing peptide coupling reaction and blowing dry; and
(t.4) washing with DMF and ethanol respectively and blowing dry to obtain said intermediate resin material of Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin,
where in said step (t), the mole number of said Boc-D-Phe-OH is 2˜5 times greater than that of said Resin and said first set of conditions as described in step (b) applies.

13. The method, as recited in claim 11, wherein in said step (v), further comprises a method of cleaving consisting the steps of:
(v.1) adding a peptide cleavage reagent at −10° C.˜30° C. (TFA/EDT/H2O/TIS=90-95/2-5/2-5/1-5, by volume) into said intermediate resin material Boc-D-Phe-Pro-Arg(Pbf)-Pro-Gly-Gly-Gly-Gly-Asn(Trt)-Gly-Asp(OtBu)-Phe-Glu(OtBu)-Glu(OtBu)-Ile-Pro-Glu(OtBu)-Glu(OtBu)-Tyr(tBu)-Leu-Resin from said step (t), allowing reaction under 10° C.˜50° C. for 1˜5 hours;
(v.2) obtaining a treated intermediate resin material by filtration under reduced pressure and evaporation;
(v.3) adding ether for precipitation such that a precipitate is formed, then collecting said precipitate and washing with ether; and
(v.4) drying said precipitate by placing said precipitate in a drying equipment under room temperature and drying in the presence of P₂O₅ under reduced pressure for 10˜14 hours to obtain said crude product of Bivalirudin;
where the concentration said intermediate resin material in said peptide cleavage reagent is 5˜50 ml/g.

14. The method, as recited in claim 11, wherein in said step (vi) further comprises the steps consisting of:
(vi.1) dissolving said crude product of Bivalirudin in an acetic acid to form a crude product solution;
(vi.2) filtering said crude product solution in step (vi.1);
(vi.3) purifying said crude product solution by flowing through C18 or C8 column, wherein the liquid phase is: 0.01˜0.5M acetate/phosphate/trifluoro-acetone acid (TFA): 10˜40% of acetonitrile (10˜60: 90˜40, by volume); the flow rate is 50˜1,500 ml/min; the detection wavelength is 250˜280 nm;
(vi.4) analyzing using a method of liquid chromatography, collecting an eluent, combining the peak obtained and removing the salt; and
(vi.5) freeze drying to obtain said final product of Bivalirudin,
where the concentration of acetic acid is 0.5˜10% and the concentration of said crude product of Bivalirudin in acetic acid is 1˜50% by weight.

15. The method, as recited in claim 11, further comprises a temperature range and a reaction time range of deprotection reaction between 10˜50° C. and 5˜60 minutes respectively.

16. The method, as recited in claim 12, further comprises a temperature range and a reaction time range of deprotection reaction between 10˜50° C. and 5˜60 minutes respectively.

17. The method, as recited in claim 13, further comprises a temperature range and a reaction time range of deprotection reaction between 10˜50° C. and 5˜60 minutes respectively.

18. The method, as recited in claim 14, further comprises a temperature range and a reaction time range of deprotection reaction between 10˜50° C. and 5˜60 minutes respectively.

19. The method, as recited in claim 11, further comprises a temperature range and a reaction time range of peptide coupling reaction between 20˜30° C. and 0.5˜5 hours respectively.

20. The method, as recited in claim 12, further comprises a temperature range and a reaction time range of peptide coupling reaction between 20˜30° C. and 0.5˜5 hours respectively.

21. The method, as recited in claim 13, further comprises a temperature range and a reaction time range of peptide coupling reaction between 20˜30° C. and 0.5˜5 hours respectively.

22. The method, as recited in claim 14, further comprises a temperature range and a reaction time range of peptide coupling reaction between 20˜30° C. and 0.5˜5 hours respectively.

23. The method, as recited in claim 11, wherein a substitution rate of said starting resin is 0.3˜1.5 mmol/g.
